(19) United States
(12) Patent Application Publication
(10) Pub. No.: US 2015/0320931 A1
(43) Pub. Date: Nov. 12, 2015

(54) LIQUID MEDICINE INJECTION DEVICE, CONTROLLER, LIQUID MEDICINE INJECTION SYSTEM, AND CONTROL METHOD

(71) Applicant: SEIKO EPSON CORPORATION, Tokyo (JP)

(72) Inventor: Yasunaga MIYAZAWA, Okaya-shi (JP)

(21) Appl. No.: 14/706,898

(22) Filed: May 7, 2015

(30) Foreign Application Priority Data
May 8, 2014 (JP) 2014-096553

(51) Int. Cl. A61M 5/142; A61M 5/50; A61M 5/172

(52) U.S. Cl. CPC A61M 5/14248; A61M 5/172; A61M 5/5086; A61M 2205/3576

(57) ABSTRACT

A liquid medicine injection device is controlled by a controller. The liquid medicine injection device includes: a liquid medicine injection unit; a first communication unit which communicates with the controller; and a control unit which switches off the communication by the first communication unit while the liquid medicine injection unit is injecting a liquid medicine. Communication between the liquid medicine injection device and the controller is carried out securely.

### BACKGROUND

1. Technical Field

The present invention relates to a liquid medicine injection device, a controller, a liquid medicine injection system, and a control method.

2. Related Art

JP-A-2013-70718 discloses a liquid medicine injection system including: a portable liquid medicine injection device that is stuck and held on a user's skin and used in this state; and a controller which transmits a signal corresponding to an input instruction from the user to the liquid medicine injection device via wireless communication. This liquid medicine injection device successively transmits the amount of injection after starting liquid medicine injection.

In the related-art liquid medicine injection system as described above, wireless communication is constantly available between the liquid medicine injection device and the controller. However, for example, in circumstances where high processing load is imposed on the arithmetic unit such as the CPU (central processing unit) of the liquid medicine injection device, an abnormality in wireless communication (such as failure to establish connection or information reception error) may occur. Such an abnormality in wireless communication causes problems such as an inability to accurately manage the amount of liquid medicine injected.

### SUMMARY

An advantage of some aspects of the invention is that communication between a liquid medicine injection device and a controller can be carried out securely.

A first aspect of the invention is directed to a liquid medicine injection device controlled by a controller and including: a liquid medicine injection unit; a communication unit which communicates with the controller; and a control unit which switches off the communication by the communication unit while the liquid medicine injection unit is injecting a liquid medicine. Thus, since the communication connection with the controller is switched off while the liquid medicine injection device is injecting the liquid medicine, for example, communication in a circumstance where high processing load is imposed on the CPU of the liquid medicine injection device can be avoided and therefore secure communication can be carried out.

In the liquid medicine injection device, the communication unit may transmit information that gives an instruction to switch off the communication, to the controller. Thus, since the instruction can be sent from the liquid medicine injection device side to the controller so as to switch off the communication from the controller side while the liquid medicine injection device is injecting the liquid medicine, the load of communication processing by the controller during the liquid medicine injection can be eliminated.

In the liquid medicine injection device, the communication unit may transmit an off-time of the communication to the controller in the case where a liquid medicine injection is started. Thus, since the off-time can be sent from the liquid medicine injection device side to the controller so as to set the off-time of the communication from the controller side while the liquid medicine injection device is injecting the liquid medicine, the communication by the controller can be switched on after the liquid medicine injection.

In the liquid medicine injection device, the control unit may switch off the communication in the case of starting liquid medicine injection, and may switch on the communication after the liquid medicine injection is finished. Thus, since the communication connection with the controller is switched on after the liquid medicine injection by the liquid medicine injection device is finished, for example, communication in a circumstance where high processing load is imposed on the CPU of the liquid medicine injection device can be avoided and therefore secure communication can be carried out.

The liquid medicine injection device may further include a notification unit which, when an abnormality in the liquid medicine injection device occurs while the communication is off, gives a notification of the occurrence of the abnormality. Thus, the user can recognize the abnormality even if the communication connection is off.

The liquid medicine injection device may include an operation unit which accepts an operation to stop the injection of the liquid medicine. Thus, the liquid medicine injection can be stopped by the operation of the liquid medicine injection device even if the liquid medicine injection cannot be stopped from the controller side because the communication connection is off.

In the liquid medicine injection device, the off-time may be a time period including a time period of one or more continuous liquid medicine injections. The communication unit may receive a setting of a length of the off-time from the controller. The control unit may decide the length of the off-time on the basis of the setting. Thus, the length of the off-time can be set according to the user's demand and the time period when the communication connection is on can be increased or decreased accordingly.

The liquid medicine injection device may operate using a battery as a power source. The off-time may be a time period including a time period of one or more continuous liquid medicine injections. The control unit may decide a length of the off-time according to a remaining capacity of the battery. Thus, for example, the time period when the communication connection is off can be increased as the remaining capacity of the battery decreases, and the operating time of the liquid medicine injection device can be thus increased.

A second aspect of the invention is directed to a controller for controlling a liquid medicine injection device, including: a communication unit which communicates with the liquid medicine injection device; and a control unit which switches off the communication by the communication unit while the liquid medicine injection device is injecting a liquid medicine. Thus, since the communication connection with the liquid medicine injection device is switched off while the liquid medicine injection device is injecting the liquid medicine, for example, communication in a circumstance where high processing load is imposed on the CPU of the liquid medicine injection device can be avoided and therefore secure communication can be carried out.

In the controller, the communication unit may receive information that gives an instruction to switch off the communication, from the liquid medicine injection device. The control unit may switch off the communication if the information that gives the instruction to switch off the communication is received. Thus, since the controller can switch off the communication with the liquid medicine injection device in accordance with the instruction from the liquid medicine injection device side, the load of communication processing during the liquid medicine injection can be eliminated.

In the controller, the communication unit may receive an off-time of the communication from the liquid medicine injection device. The control unit may switch off the communication during the off-time, and may switch on the communication after the off-time. Thus, the controller can switch on the communication after the liquid medicine injection, in accordance with the off-time from the liquid medicine injection device side.

The controller may include an operation unit which accepts a setting of a length of the off-time. The communication unit may transmit the setting to the liquid medicine injection device. Thus, the length of the off-time can be set according to the user's demand and the time period when the communication connection is on can be increased or decreased accordingly.

The controller may include a notification unit that notifies that the communication is shifted to an off-state or returned to an on-state. Thus, the user can recognize whether the liquid medicine injection device is available for operation or not.

The controller may include an operation unit which accepts a setting of the liquid medicine injection device, and a notification unit which notifies that the communication is in an off-state if the setting of the liquid medicine injection device is accepted while the communication is off. The user can recognize that the liquid medicine injection by the liquid medicine injection device is underway and that the setting of an injection program or the like will not be sent to the liquid medicine injection device.

A third aspect of the invention is directed to a liquid medicine injection system including a liquid medicine injection device and a controller which controls the liquid medicine injection device. The liquid medicine injection device includes: a liquid medicine injection unit; a first communication unit which communicates with the controller; and a first control unit which switches off the communication by the first communication unit while the liquid medicine injection device is injecting a liquid medicine. The controller includes: a second communication unit which communicates with the liquid medicine injection device; and a second control unit which switches off the communication by the second communication unit while the liquid medicine injection device is injecting the liquid medicine. Thus, since the communication connection between the controller and the liquid medicine injection device is switched off while the liquid medicine injection device is injecting the liquid medicine, for example, communication in a circumstance where high processing load is imposed on the CPU of the liquid medicine injection device can be avoided and therefore secure communication can be carried out.

A fourth aspect of the invention is directed to a control method for a liquid medicine injection device which communicates with a controller, including: switching off the communication with the controller while the liquid medicine injection device is injecting a liquid medicine. Thus, since the communication connection with the controller is switched off while the liquid medicine injection device is injecting the liquid medicine, for example, communication in a circumstance where high processing load is imposed on the CPU of the liquid medicine injection device can be avoided and therefore secure communication can be carried out.

### BRIEF DESCRIPTION OF THE DRAWINGS

The invention will be described with reference to the accompanying drawings, wherein like numbers reference like elements.

FIG. 1 shows an example of the configuration of a liquid medicine injection system according to an embodiment of the invention.

FIG. 2 shows an example of the configuration of a liquid medicine injection device and a controller.

FIGS. 3A and 3B illustrate a mechanical structure involved in a liquid medicine injection, in the liquid medicine injection device.

FIG. 4 illustrates a mechanical structure involved in a liquid medicine injection, in the liquid medicine injection device.

FIG. 5 is a flowchart showing an example of an operation of the controller.

FIG. 6 is a flowchart showing an example of an operation of the liquid medicine injection device.

FIG. 7 illustrates an example of a wireless connection state in a bolus injection.

FIG. 8 illustrates an example of a wireless connection state in a basal injection.

FIG. 9 is a sequence diagram showing an example of a flow of processing in the case of carrying out a basal injection.

FIG. 10 is a sequence diagram showing an example of a flow of processing in the case of carrying out a bolus injection during a basal injection.

FIG. 11 is a flowchart showing a modification of the operation of the controller.

FIG. 12 illustrates a modification of the wireless connection state in the basal injection.

### DESCRIPTION OF EXEMPLARY EMBODIMENTS

Hereinafter, an embodiment of the invention will be described with reference to the drawings.

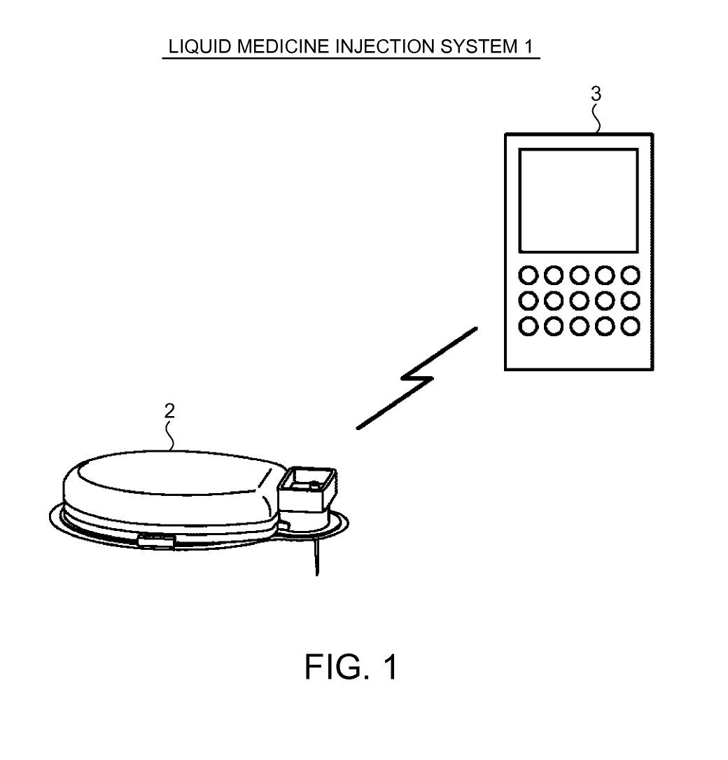

FIG. 1 shows an example of the configuration of a liquid medicine injection system according an embodiment of the invention.

A liquid medicine injection system 1 includes a liquid medicine injection device 2 and a controller 3. The liquid medicine injection device 2 and the controller 3 are connectable to each other via short-range wireless communication such as Bluetooth (trademark registered), for example.

The liquid medicine injection device 2 is, for example, a micro pump that is stuck on an abdominal part or the like of the user and injects a liquid medicine such as insulin. The liquid medicine injection device 2 has a cartridge section which stores the liquid medicine, a hollow needle member which feeds the liquid medicine into a living body, a pump section which sucks the liquid medicine from the storage section and ejects the liquid medicine to the needle member, and a power-supply section such as a battery, or the like.

The liquid medicine injection device 2 also carries out, for example, start of a liquid medicine injection, injection of the liquid medicine, stop of the liquid medicine injection, and the like, according to an injection program transmitted from the controller 3. The injection program includes, for example, an instruction to start a bolus injection, the amount of liquid medicine and time for the bolus injection, an instruction to stop the bolus injection, an instruction to start a basal injection, the amount of liquid medicine and time for basal injection, an instruction to stop the basal injection, and the like. Here, the injection program is a general term that refers to an aggregate of commands and parameters and not program codes themselves (source codes or the like).

The controller 3 is, for example, a mobile computer or the like. The controller 3, for example, accepts a setting of an injection program from the user and transmits the set injection program to the liquid medicine injection device 2.

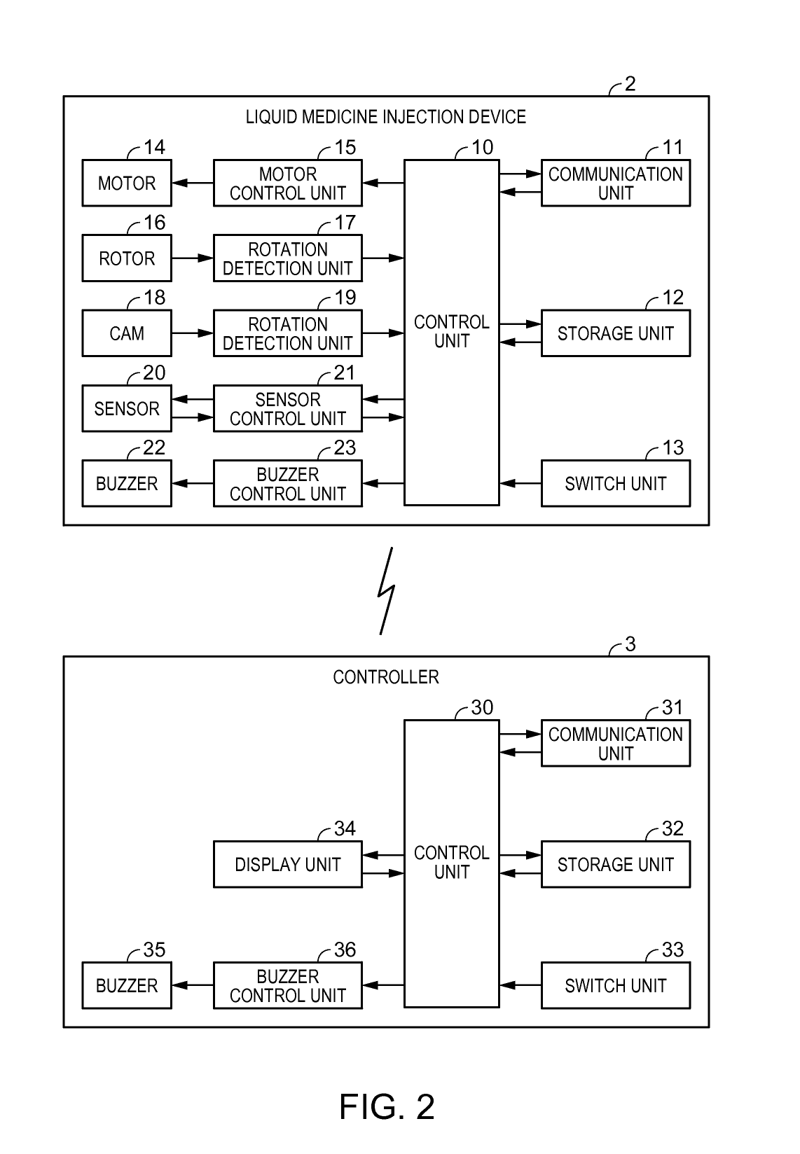

FIG. 2 shows an example of the configuration of the liquid medicine injection device and the controller.

The liquid medicine injection device 2 has a control unit 10, a communication unit 11, a storage unit 12, a switch unit 13, a motor 14, a motor control unit 15, a rotor 16, a rotation detection unit 17, a cam 18, a rotation detection unit 19, a sensor 20, a sensor control unit 21, a buzzer 22, a buzzer control unit 23, and the like. The control unit 10 is equivalent to the first control unit according to the invention. The communication unit 11 is equivalent to the first communication unit according to the invention. The switch unit 13 is equivalent to the operation unit according to the invention. The buzzer 22 is equivalent to the notification unit according to the invention. The liquid medicine injection device 2 may has a light source unit such as an LED (light emitting diode), as the notification unit.

The control unit 10 has, for example, a CPU (central processing unit), a RAM (random access memory) used for temporary storage of various data, and the like. The control unit 10 operates according to a control program stored in the storage unit 12 and thereby centrally controls the operation of the liquid medicine injection device 2.

For example, as the control unit 10 receives an injection program from the controller 3 via the communication unit 11, described later, the control unit 10 stores the received injection program in the storage unit 12, described later. The control unit 10 also sends an instruction to the motor control unit 15, described later, on the basis of an injection program of bolus injection or basal injection stored in the storage unit 12, and thereby controls the bolus injection or basal injection to a living body. In a bolus injection, for example, a predetermined amount of a liquid medicine is injected continuously for a predetermined time period (for example, 15 minutes). In a basal injection, for example, a predetermined amount of a liquid medicine is injected intermittently, each time for a predetermined time period (for example, several seconds), during a predetermined time period (for example, 24 hours). During each of the intermittent injection time periods of the basal injection, the liquid medicine is injected continuously. The control of the liquid medicine injection is not a main feature of the invention and therefore will not be described further in detail.

While executing the injection program, for example, the control unit 10 switches off the wireless connection of the communication unit 11 during a liquid medicine injection. The processing involved in the switching on and off of the wireless connection will be described in detail below. The switching on and off of the wireless connection can also be regarded as the switching on and off of communication.

The communication unit 11 is, for example, a communication interface which controls short-range wireless communication such as Bluetooth, and performs wireless connection and communication with an external device such as the controller 3. The communication unit 11 transmits information inputted from the control unit 10, to the external device, and outputs information received from the external device, to the control unit 10.

The storage unit 12 is made up of, for example, a non-volatile memory such as a mask ROM (read only memory), flash memory, or FeRAM (ferroelectric RAM or ferroelectric memory). In the storage unit 12, a control program and various data or the like for controlling the operation of the liquid medicine injection device 2 are stored.

The switch unit 13 has operation keys to give various instructions to the liquid medicine injection device 2. As the user operates an operation key, the switch unit 13 outputs an operation signal corresponding to the content of the operation by the user, to the control unit 10. The operation keys include, for example, a “power key” to switch on and off the power source, a “stop key” to stop a liquid medicine injection, and the like. The stop key enables the user to stop a liquid medicine injection even if the wireless connection is off (where an operation from the controller 3 is not available).

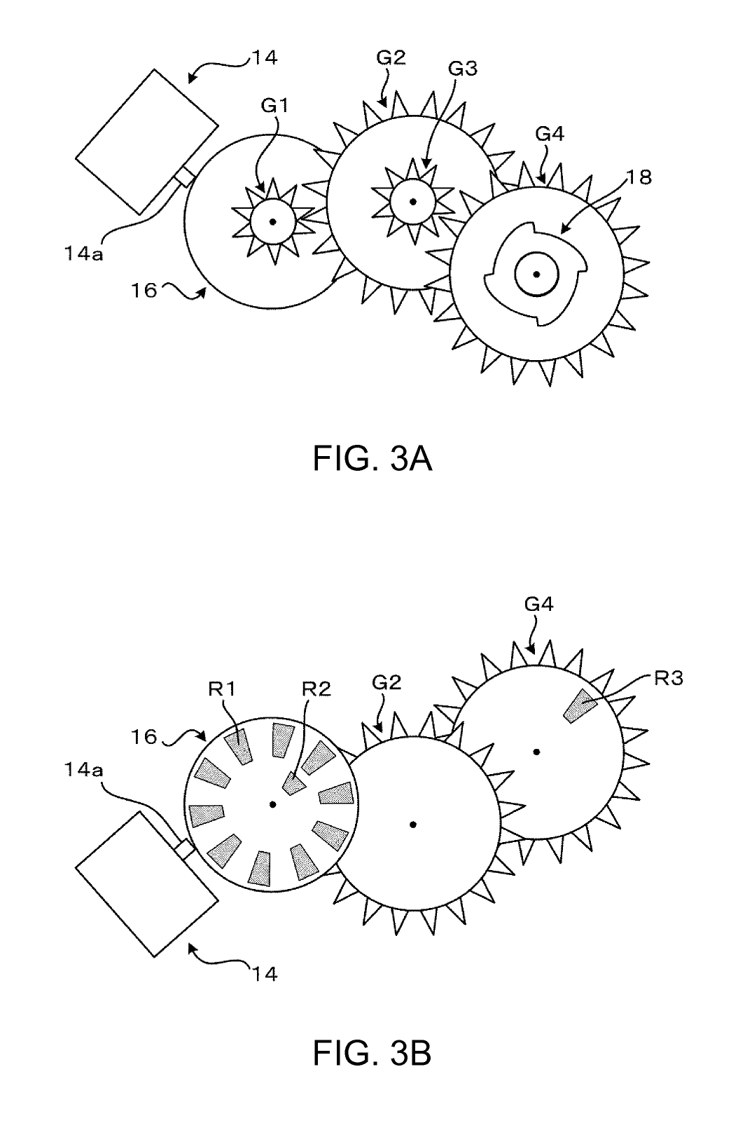

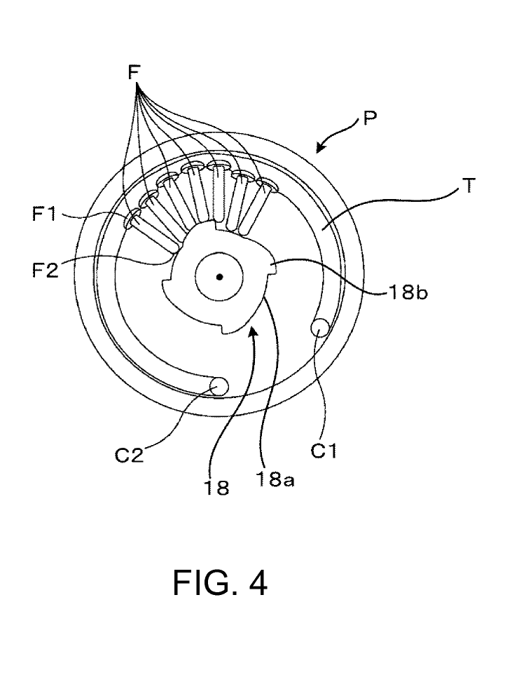

The mechanical structure for liquid medicine injection including the motor 14, the rotor 16 and the cam 18 will be described with reference to FIGS. 3A and 3B and FIG. 4. FIGS. 3A and 3B and FIG. 4 illustrate the mechanical structure involved in a liquid medicine injection, in the liquid medicine injection device. FIG. 3A shows the rotor 16 and the like, as viewed from the front side. FIG. 3B shows the rotor 16 and the like, as viewed from the back side.

The motor 14 is a motor for providing a rotational drive force to the cam 18, described later, via the rotor 16 and various gears G, described later. The motor 14 is a piezoelectric motor, for example, and has a plate-like member and a spring (neither of them shown). The spring with an elastic force thereof urges the plate-like member toward the rotor 16. A distal end part 14a of the plate-like member contacts the circumferential surface of the rotor 16. The plate-like member has a piezoelectric body and two electrodes and changes shape according to a change in the voltage applied to the two electrodes. For example, vertical oscillation and bending oscillation are alternately generated in the plate-like member according to the voltage applied, and the shape of the plate-like member is thus changed so as to rotate the rotor 16 in a predetermined direction.

The rotor 16 is made to rotate in a predetermined direction by the plate-like member of the motor 14. The rotor 16 has a pinion gear G1 which rotates in unison with the rotor 16. The pinion gear G1 engages with a gear G2 and causes the gear G2 to rotate in a predetermined direction. The gear G2 has a pinion gear G3 which rotates in unison with the gear G2. The pinion gear G3 engages with a gear G4 and causes the gear G4 to rotate in a predetermined direction. The gear G4 has a cam 18 which rotates in unison with the gear G4. Thus, the motive power from the motor 14 is transmitted to the cam 18.

A reflection member R1 and a reflection member R2 are provided on the back side of the rotor 16. A plurality of the reflection members R1 is provided with a predetermined space from each other along the circumference of the rotor 16. The reflection members R1 are used by the rotation detection unit 17, described later, to detect the rotation angle of the rotor 16. The reflection member R2 is used by the rotation detection unit 17, described later, to detect a reference angle of the rotation of the rotor 16. Meanwhile, a reflection member R3 is provided on the backside of the gear G4. The reflection member R3 is used by the rotation detection unit 19, described later, to detect a reference angle of the rotation of the cam 18.

The cam 18 is inserted in a pump section P. The pump section P has a finger F, a tube T, a connector C1, and a connector C2. The connector C1 is a connector to suck a liquid medicine into the pump section P from a storage section. The connector C2 is a connector to eject the liquid medicine to a needle member from inside the pump section P. In the pump section P, an arc-shaped tube guide groove (not shown) for guiding the tube T is formed, and the tube T is accommodated in the tube guide groove. The tube guide groove is formed on the circumference of a circle formed about the rotation axis of the cam 18 as the center thereof. One end of the tube T is connected with the connector C1, and the other end is connected with the connector C2.

On the inner side of the arc of the tube guide groove, a plurality of finger guides (not shown) is formed, and a finger F is accommodated in each of the finger guides. The finger guides are formed on straight lines that connect the rotation axis of the cam 18 to the arc of the tube guide groove. Thus, forward ends F1 of the fingers F are arranged to abut substantially perpendicularly against the tube T, and rear ends F2 of the fingers F are arranged to face a cam surface 18a of the cam 18.

As the cam 18 rotates, the cam surface 18a abuts against the rear ends F2 of the fingers F and can cause the fingers F to slide thereon. Four cam ridges 18b are formed on the cam 18. Each cam ridge 18b has a shape that gradually increases in height from the lowest part of the cam ridge to the highest part, and on reaching the highest part, falls to the lowest part of the next cam ridge. Thus, as the cam 18 rotates, the forward ends F1 of the plurality of fingers F press the tube T sequentially in a direction from the connector C1 side to the connector C2 side. The liquid medicine in the tube T can be feed from the connector C1 side to the connector C2 side. Thus, the liquid medicine is fed from the storage section to the needle member via the pump section P and then injected into a living body.

The liquid medicine injection unit according to the invention is equivalent to, for example, the mechanical structure for liquid medicine injection including the motor 14 and the like.

Back to the explanation of FIG. 2, the motor control unit 15 changes the voltage applied to the two electrodes of the plate-like member of the motor 14 in accordance with an instruction from the control unit 10.

The rotation detection unit 17 has a light source, a light sensor and the like. The rotation detection unit 17 switches on the light source to emit light to the reflection member R1 and the reflection member R2, and causes the light sensor to detect the reflected light from the reflection member R1 and the reflection member R2. The rotation detection unit 17 also detects the rotation angle and the number of rotations of the rotor 16 on the basis of the reflected light from the reflection member R1 and the reflection member R2 that is detected, and outputs the rotation angle and the number of rotations to the control unit 10. The rotation angle and the number of rotations of the rotor 16 are used for feedback control or the like of the motor 14 by the control unit 10.

The rotation detection unit 19 has a light source, a light sensor and the like. The rotation detection unit 19 switches on the light source to emit light to the reflection member R3, and causes the light sensor to detect the reflected light from the reflection member R3. The rotation detection unit 19 also detects the number of rotations of the cam 18 on the basis of the reflected light from the reflection member R3 that is detected, and outputs the number of rotations to the control unit 10. The number of rotations of the cam 18 is used for control of the amount of the liquid medicine injected or the like by the control unit 10.

The sensor 20 is, for example, a temperature sensor which detects temperature, a pressure sensor which detects pressure inside the tube T, a light sensor which detects air bubbles in the liquid medicine, or the like.

The sensor control unit 21 controls the sensor 20, acquires a value indicating temperature, a value indicating pressure, a value indicating the amount of air bubbles, or the like, from the sensor 20, and outputs the value to the control unit 10. These pieces of information are used for abnormality detection in the liquid medicine injection device 2 by the control unit 10.

The buzzer 22 is an audio output device which outputs a sound that notifies the user of an abnormality.

The buzzer control unit 23 causes the buzzer 22 to output a sound in accordance with an instruction from the control unit 10.

The controller 3 has a control unit 30, a communication unit 31, a storage unit 32, a switch unit 33, a display unit 34, a buzzer 35, a buzzer control unit 36, and the like. The control unit 30 is equivalent to the second control unit according to the invention. The communication unit 31 is equivalent to the second communication unit according to the invention. The switch unit 33 is equivalent to the operation unit according to the invention. The display unit 34 and the buzzer 35 are equivalent to the notification unit according to the invention. The controller 3 may include a light source unit such as an LED (light emitting diode), as the notification unit.

The control unit 30 has, for example, a CPU, a RAM used for temporary storage of various data, and the like. The control unit 30 operates according to a control program stored in the storage unit 32 and thereby centrally controls the operation of the controller 3.

For example, the control unit 30 accepts a setting of an injection program from the user via the switch unit 33, described later. The injection program includes, for example, a setting about a bolus injection (for example, the amount of liquid medicine and the time period of the bolus injection, an instruction to start the bolus injection, an instruction to stop the bolus injection, and the like), and a setting about a basal injection (for example, the amount of liquid medicine and the time period of the basal injection, an instruction to start the basal injection, an instruction to stop the basal injection, and the like). Also, the control unit 30 transmits the accepted injection program to the liquid medicine injection device 2 via the communication unit 31. The processing to generate the injection program is not a main feature of the invention and therefore will not be described further in detail.

In the case where an instruction to switch off the wireless connection is received from the liquid medicine injection device 2 via the communication unit 31, for example, the control unit 30 switches off the wireless connection of the communication unit 31. The processing involved in the switching on and off of the wireless connection will be described in detail below. The switching on and off of the wireless connection can also be regarded as the switching on and off of communication.

The communication unit 31 is, for example, a communication interface which controls short-range wireless communication such as Bluetooth, and performs wireless connection and communication with an external device such as the liquid medicine injection device 2. The communication unit 31 transmits information inputted from the control unit 30, to the external device, and outputs information received from the external device, to the control unit 30.

The storage unit 32 is made up of, for example, a non-volatile memory such as a mask ROM, flash memory, or FeRAM. In the storage unit 32, a control program and various data or the like for controlling the operation of the controller 3 are stored.

The switch unit 33 has operation keys to give various instructions to the controller 3. As the user operates an operation key, the switch unit 33 outputs an operation signal corresponding to the content of the operation by the user, to the control unit 30. The operation keys include, for example, a “power key” to switch on and off the power source, a “menu key” to display a menu image for carrying out various settings, a “cursor key” used for selection of an item on the menu image or the like, an “enter key” to finalize a selected item, a “cancel key” used for cancelation of an operation, and the like. By operating various keys, a setting about a bolus injection program (for example, the amount of liquid medicine and the time period of the bolus injection, an instruction to start the bolus injection, an instruction to stop the bolus injection, and the like), and a setting about a basal injection program (for example, the amount of liquid medicine and the time period of the basal injection, an instruction to start the basal injection, an instruction to stop the basal injection, and the like) are inputted.

The display unit 34 is, for example, a liquid crystal display, organic EL (electro-luminescence) display, or the like. The display unit 34 displays an operation screen, a message, a dialogue or the like in accordance with an instruction from the control unit 30.

The buzzer 35 is an audio output device which outputs a sound that notifies the user of an abnormality.

The buzzer control unit 36 causes the buzzer 35 to output a sound in accordance with an instruction from the control unit 30.

Next, the operations of the controller 3 and the liquid medicine injection device 2 will be described.

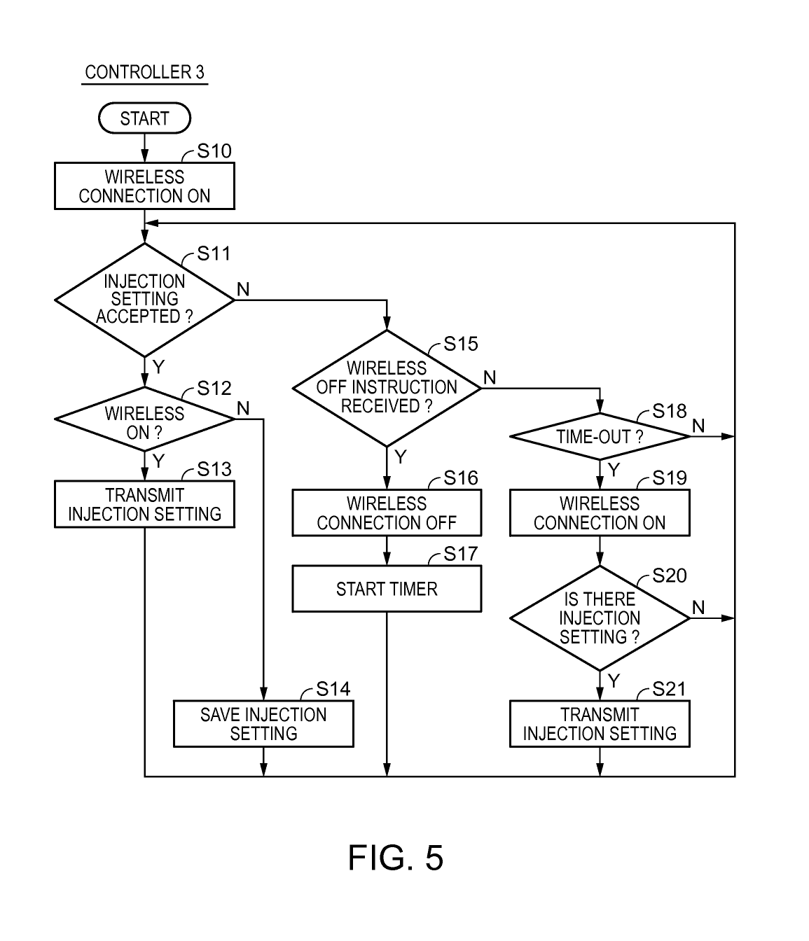

FIG. 5 is a flowchart showing an example of the operation of the controller. This flowchart starts, for example, when the power source of the controller 3 is switched on. It is also assumed that the power source of the liquid medicine injection device 2 is switched on.

First, the control unit 30 switches on the wireless connection of the communication unit 31 (Step S10). Thus, the controller 3 enters a wireless connection on-state and is enabled to communicate with the liquid medicine injection device 2.

Then, the control unit 30 determines whether a setting of an injection program is accepted or not (Step S11). For example, the control unit 30 determines whether a setting of an injection program is inputted via the switch unit 33 or not. The injection program inputted here includes, for example, the amount of liquid medicine and the time period of a bolus injection or basal injection, and an instruction to start the bolus injection or basal injection.

If it is determined that a setting of an injection program is accepted (Y in Step S11), the control unit 30 determines whether the wireless connection state of the communication unit 31 is on or off (Step S12). If it is determined that the wireless connection state is on (Y in Step S12), the control unit 30 transmits the injection program accepted in Step S11 to the liquid medicine injection device 2 via the communication unit 31 (Step S13) and carries out the processing of Step S11 again. If it is determined that the wireless connection state is off (N in Step S12), the control unit 30 saves the injection program accepted in Step S11, in the storage unit 32 (Step S14), and carries out the processing of Step S11 again.

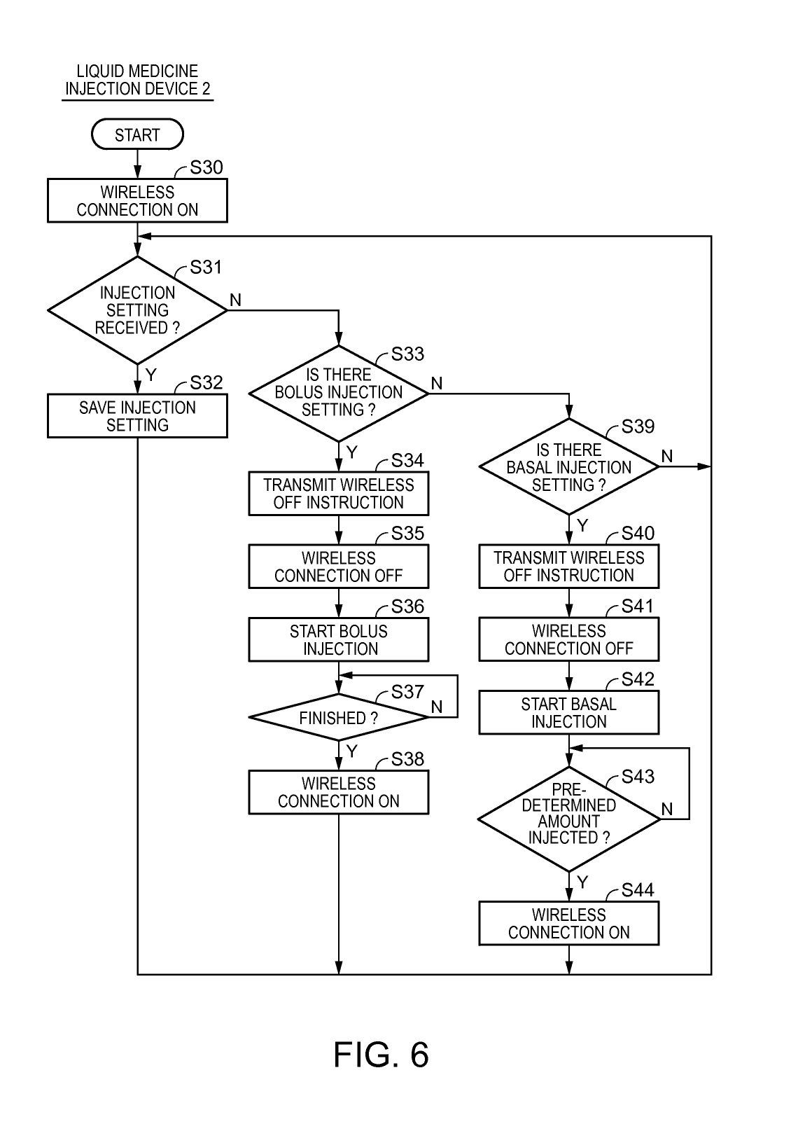

If it is determined that a setting of an injection program is not accepted (N in Step S11), the control unit 30 determines whether a wireless off instruction is received via the communication unit 31 or not (Step S15). In the embodiment, the wireless off instruction includes information that gives an instruction to switch off wireless connection, and an off-time of the wireless connection. As will be described later with reference to FIG. 6, the wireless off instruction is transmitted from the liquid medicine injection device 2 at the timing when the liquid medicine injection device 2 starts a liquid medicine injection.

If it is determined that a wireless off instruction is received (Y in Step S15), the control unit 30 switches off the wireless connection of the communication unit 31 (Step S16). Thus, the controller 3 enters a wireless connection off state and is disabled from communicating with the liquid medicine injection device 2. The control unit 30 also starts the timer (Step S17) and carries out the processing of Step S11 again. In Step S18, described later, the control unit 30 monitors the lapse of the off-time of the wireless connection received in Step S15, using the timer started in Step S17.

If it is determined that a wireless off instruction is not received (N in Step S15), the control unit 30 determines whether the off-time of the wireless connection has reached a time-out or not (Step S18). If it is determined that it is not a time-out (N in Step S18), the control unit 30 carries out the processing of Step S11 again.

If it is determined that it is a time-out (Y in Step S18), the control unit 30 switches on the wireless connection of the communication unit 31 (Step S19). Thus, the controller 3 enters a wireless connection on-state and is enabled again to communicate with the liquid medicine injection device 2.

Then, the control unit 30 determines whether there is a setting of an injection program or not (Step S20). For example, the control unit 30 determines whether an injection program is saved in the storage unit 32 or not. If it is determined that there is no setting of an injection program (N in Step S20), the control unit 30 carries out the processing of Step S11 again. If it is determined that there is a setting of an injection program (Y in Step S20), the control unit 30 acquires the injection program from the storage unit 32, transmits the injection program to the liquid medicine injection device 2 via the communication unit 31 (Step S21), and carries out the processing of Step S11 again. In the case where the processing of Step S14 is carried out, the injection program is saved in the storage unit 32.

FIG. 6 is a flowchart showing an example of the operation of the liquid medicine injection device. This flowchart is started, for example, when the power source of the liquid medicine injection device 2 is switched on. It is also assumed that the power source of the controller 3 is switched on.

First, the control unit 10 switches on the wireless connection of the communication unit 11 (Step S30). Thus, the liquid medicine injection device 2 enters a wireless connection on-state and is enabled to communicate with the controller 3.

Then, the control unit 10 determines whether a setting of an injection program is received or not (Step S31). For example, the control unit 10 determines whether an injection program is received from the controller 3 via the communication unit 11 or not. The injection program received here includes, for example, the amount of liquid medicine and the time period of a bolus injection or basal injection, and an instruction to start the bolus injection or basal injection.

If it is determined that an injection program is received (Y in Step S31), the control unit 10 saves the injection program received in Step S31, in the storage unit 12 (Step S32), and carries out the processing of Step S31 again.

If it is determined that an injection program is not received (N in Step S31), the control unit 10 determines whether there is a setting of a bolus injection or not (Step S33). For example, the control unit 10 determines whether an injection program of a bolus injection (unexecuted injection program) is saved in the storage unit 12 or not.

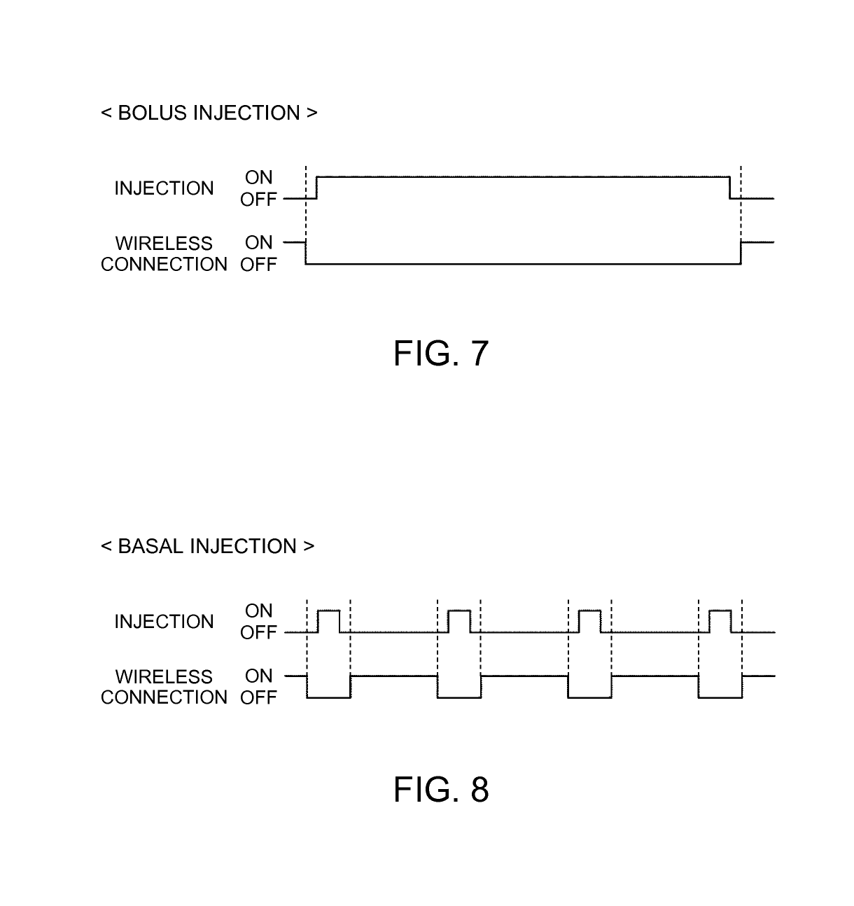

If it is determined that there is a setting of an injection program of a bolus injection (Y in Step S33), the control unit 10 generates a wireless off instruction and transmits the wireless off instruction to the controller 3 via the communication unit 11 (Step S34). The wireless off instruction includes information that gives an instruction to switch off wireless connection, and the off-time of the wireless connection. In the embodiment, the off time of the wireless connection in a bolus injection is a time period (time period during which the wireless connection is off) including a time period from the start to the end of the bolus injection (time period during which the injection is on), for example, as shown in FIG. 7 (illustrating an example of the wireless connection state in a bolus injection).

Then, the control unit 10 switches off the wireless connection of the communication unit 11 (Step S35). Thus, the liquid medicine injection device 2 enters a wireless connection off-state and is disabled from communicating with the controller 3.

Subsequently, the control unit 10 sends an instruction to the motor control unit 15 to start the bolus injection on the basis of the injection program of the bolus injection (Step S36). Then, the control unit 10 determines whether the bolus injection is finished or not (Step S37). If it is determined that the bolus injection is not finished (N in Step S37), the control unit 10 continues the bolus injection. Whether the bolus injection is finished or not may be determined, for example, by determining whether the injection of a predetermined amount of liquid medicine is finished or not.

If it is determined that the bolus injection is finished (Y in Step S37), the control unit 10 switches on the wireless connection of the communication unit 11 (Step S38) and carries out the processing of Step S31 again. Thus, the liquid medicine injection device 2 enters a wireless connection on-state and is enabled to communicate with the controller 3 again.

If it is determined that there is no setting of an injection program of a bolus injection (N in Step S33), the control unit 10 determines whether there is a setting of a basal injection or not (Step S39). For example, the control unit 10 determines whether an injection program of a basal injection (unexecuted injection program or injection program currently being executed) is saved in the storage unit 12 or not. If it is determined that there is no setting of an injection program of a basal injection (N in Step S39), the control unit 10 carries out the processing of Step S31 again.

If it is determined that there is a setting of an injection program of a basal injection (Y in Step S39), the control unit 10 generates a wireless off instruction and transmits the wireless off instruction to the controller 3 via the communication unit 11 (Step S40). The wireless off instruction includes information that gives an instruction to switch off wireless connection, and the off-time of the wireless connection. In the embodiment, the off-time of the wireless connection in a basal injection is a time period (each time period during which the wireless connection is off) including at least one or more of respective intermittent injection time periods (respective time periods during which the injection is on) of the basal injection, for example, as shown in FIG. 8 (illustrating an example of the wireless connection state in a basal injection). In FIG. 8, one off-time period of the wireless connection includes one injection time period.

Then, the control unit 10 switches off the wireless connection of the communication unit 11 (Step S41). Thus, the liquid medicine injection device 2 enters a wireless connection off state and is disabled from communicating with the controller 3.

Subsequently, the control unit 10 sends an instruction to the motor control unit 15 to start the basal injection on the basis of the injection program of the basal injection (Step S42). Then, the control unit 10 determines whether a predetermined amount of the basal injection is finished or not (Step S43). For example, in FIG. 8, one injection is equivalent to the predetermined amount. If it is determined that a predetermined amount of the basal injection is not finished (N in Step S43), the control unit 10 continues the basal injection.

If it is determined that a predetermined amount of the basal injection is finished (Y in Step S43), the control unit 10 switches on the wireless connection of the communication unit 11 (Step S44) and carries out the processing of Step S31 again. Thus, the liquid medicine injection device 2 enters a wireless connection on-state and is enabled to communicate with the controller 3 again.

In Step S39, if there is an injection program that is currently being executed, the control unit 10 may determine whether a predetermined time (for example, equivalent to the interval between the times when the wireless connection is switched off in FIG. 8) has passed or not from when the wireless connection is switched on in the previous basal injection of the predetermined amount (that is, whether the next basal injection time of the predetermined amount has come or not). The control unit 10 determines that it is Y in Step S39 if the predetermined time has passed, and N in Step S39 if the predetermined time has not passed.

As shown in the flowcharts of FIGS. 5 and 6, the liquid medicine injection device 2 switches off the wireless connection in the case of starting a liquid medicine injection, and the controller 3 switches off the wireless connection in accordance with an instruction from the liquid medicine injection device 2. Meanwhile, the liquid medicine injection device 2 switches on the wireless connection in the case where the liquid medicine injection is finished. The controller 3 switches on the wireless connection in the case where the off-time included in the instruction from the liquid medicine injection device 2 has passed.

In the case where the controller 3 receives a setting of an injection program in the state where the wireless connection is off, the controller 3 saves the setting in the storage unit. After the wireless connection returns to an on-state, the controller 3 transmits the setting to the liquid medicine injection device 2. When the wireless connection returns to an on-state, the liquid medicine injection device 2 receives the setting of the injection program. The liquid medicine injection device 2 gives priority to the execution of a bolus injection over that of a basal injection.

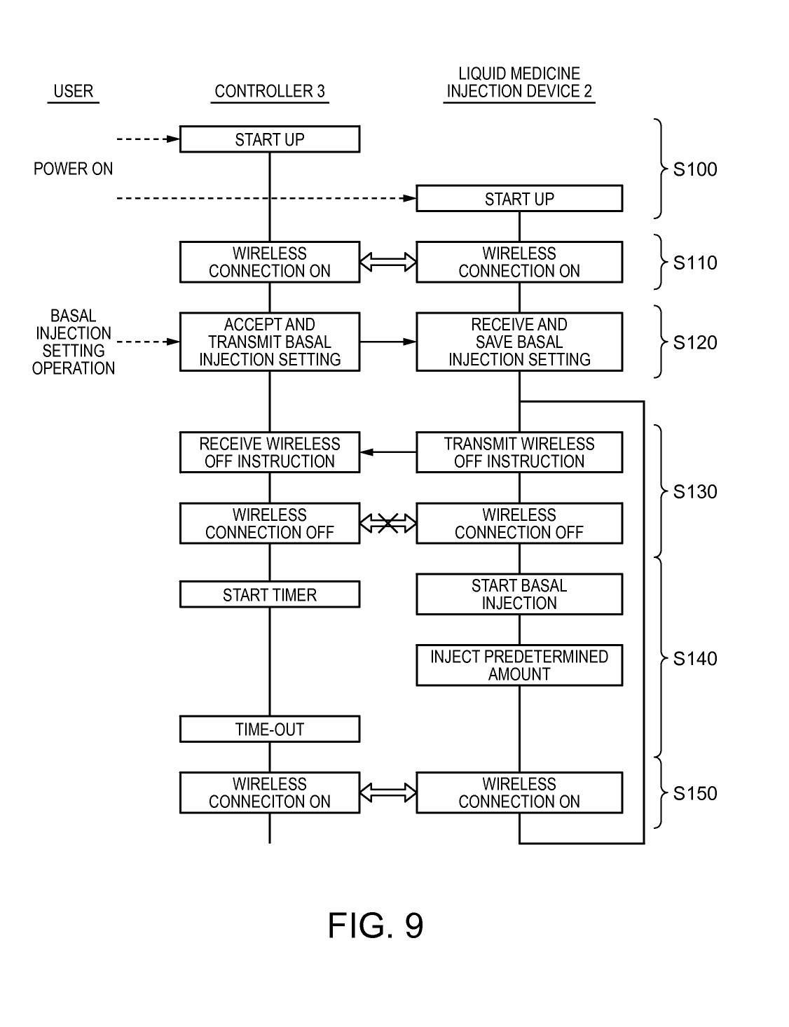

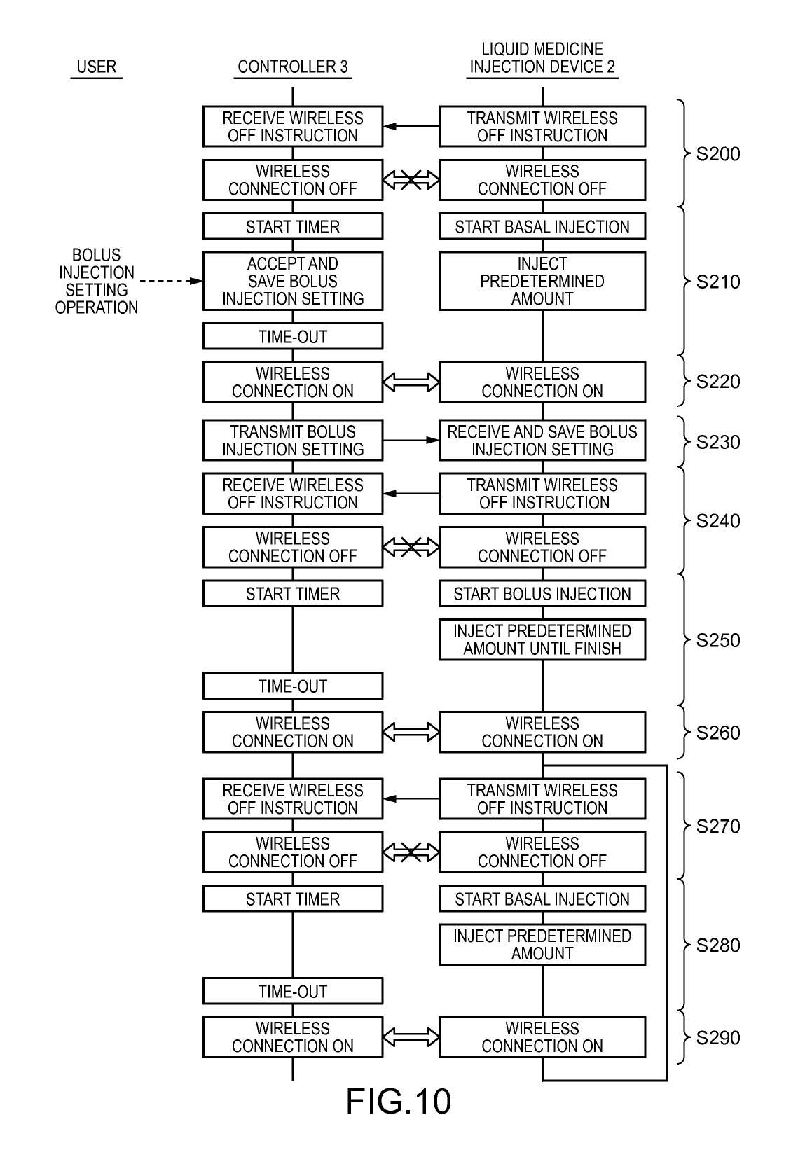

Next, an example of processing realized by the operations of the controller 3 and the liquid medicine injection device 2 will be described with reference to FIGS. 9 and 10.

FIG. 9 is a sequence diagram showing an example of a flow of processing in the case of carrying out a basal injection.

First, the controller 3 and the liquid medicine injection device 2 start up as the power is turned on by the user (Step S100). Then, the controller 3 and the liquid medicine injection device 2 switch on wireless connection and are thus enabled to communicate with each other (Step S110).

Then, the controller 3 accepts a setting of an injection program of a basal injection from the user and transmits the setting to the liquid medicine injection device 2 (Step S120). The liquid medicine injection device 2 receives the setting of the injection program of the basal injection from the controller 3 and saves the setting in the storage unit (Step S120). Subsequently, the processing of Steps S130 to S150 is repeated.

First, the liquid medicine injection device 2 transmits a wireless connection off instruction to the controller 3 and switches off the wireless connection (Step S130). The liquid medicine injection device 2 also starts a basal injection based on the injection program and injects a predetermined amount (Step S140). Then, the liquid medicine injection device 2 switches on the wireless connection (Step S150).

Meanwhile, the controller 3 receives the wireless connection off instruction from the liquid medicine injection device 2 and switches off the wireless connection (Step S130). The controller 3 also starts the timer and reaches a time-out as the off-time of the wireless connection included in the wireless connection off instruction passes (Step S140). Then, the controller 3 switches on the wireless connection (Step S150).

FIG. 10 is a sequence diagram showing an example of a flow of processing in the case of carrying out a bolus injection during a basal injection. In the liquid medicine injection device 2, an injection program of a basal injection is saved and being executed.

The liquid medicine injection device 2 transmits a wireless connection off instruction to the controller 3 and switches off the wireless connection (Step S200). The liquid medicine injection device 2 also starts a basal injection based on the injection program and injects a predetermined amount (Step S210). Then, the liquid medicine injection device 2 switches on the wireless connection (Step S220).

Meanwhile, the controller 3 receives the wireless connection off instruction from the liquid medicine injection device 2 and switches off the wireless connection (Step S200). The controller 3 also starts the timer and reaches a time-out as the off-time of the wireless connection included in the wireless connection off instruction passes (Step S210). Here, after the start of the timer and before the time-out, the controller 3 accepts a setting of an injection program of a bolus injection from the user and saves the setting in the storage unit 32. Then, the controller 3 switches on the wireless connection (Step S220).

As the wireless connection is switched on, the controller 3 transmits the injection program of the bolus injection saved in the storage unit 32 to the liquid medicine injection device 2 (Step S230). The liquid medicine injection device 2 receives the setting of the injection program of the bolus injection from the controller 3 and saves the setting in the storage unit 12 (Step S230). Subsequently, the processing of Steps S240 to S260 is repeated.

First, the liquid medicine injection device 2 transmits a wireless connection off instruction to the controller 3 and switches off the wireless connection (Step S240). The liquid medicine injection device 2 also starts a bolus injection based on the injection program and injects a predetermined amount until the bolus injection is finished (Step S250). Then, the liquid medicine injection device 2 switches on the wireless connection (Step S260).

Meanwhile, the controller 3 receives the wireless connection off instruction from the liquid medicine injection device 2 and switches off the wireless connection (Step S240). The controller 3 also starts the timer and reaches a time-out as the off-time of the wireless connection included in the wireless connection off instruction passes (Step S250). Then, the controller 3 switches on the wireless connection (Step S260).

After the bolus injection is finished as described above, the processing of the basal injection is executed again (the processing of Steps S270 to S290 is repeated). The processing of Steps S270 to S290 is similar to Steps S130 to S150 in FIG. 9 and therefore will not be described further in detail.

The one embodiment of the invention is described above. According to this embodiment, communication between the liquid medicine injection device and the controller can be carries out securely. That is, in the embodiment, while the liquid medicine injection device is injecting the liquid medicine, the wireless connection is switched off. If the liquid medicine injection device is not injecting the liquid medicine, the wireless connection is switched on. Thus, the setting of the injection program can be transmitted from the controller, for example, avoiding a circumstance where high processing load is imposed on the CPU of the liquid medicine injection device. Therefore, more secure communication can be carried out.

Of course, the embodiment of the invention is intended to illustrate but not limit the gist and scope of the invention. For example, the embodiment may be modified as follows.

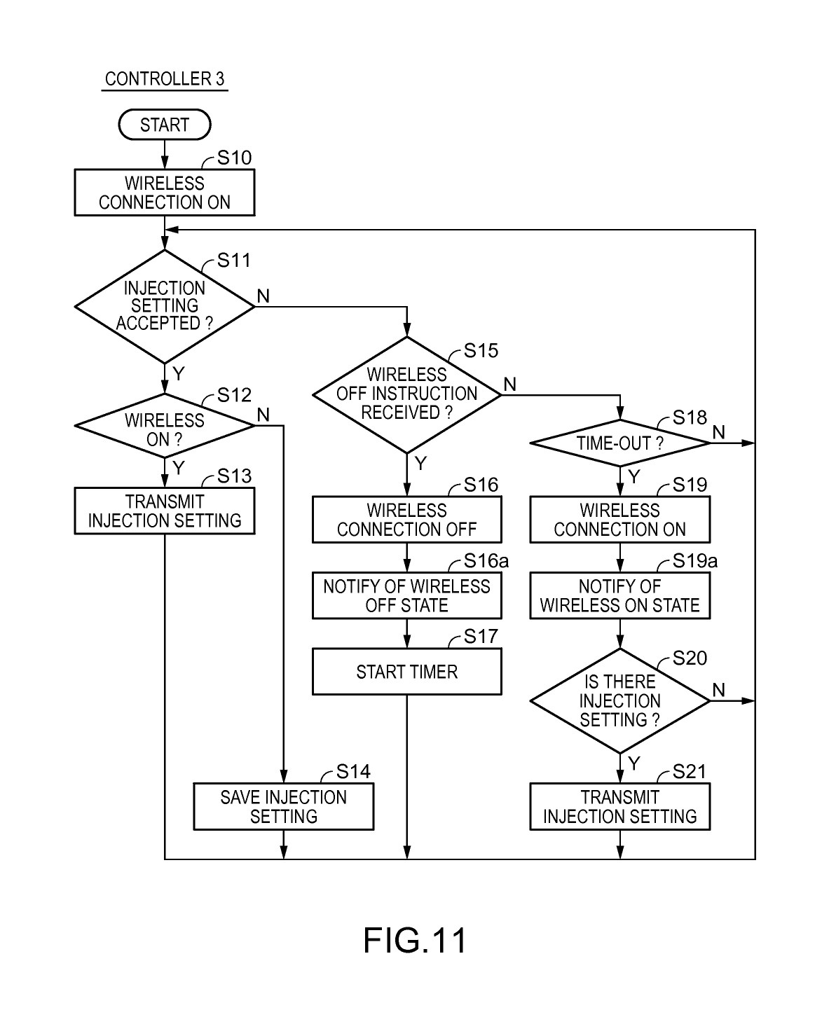

FIG. 11 is a flowchart showing a modification of the operation of the controller. In this modification, the user is notified of the wireless connection state. In FIG. 11, the processing denoted by the same reference numbers as in FIG. 5 is similar to that of FIG. 5 and therefore will not be described further.

After the processing of Step S16, the control unit 30 notifies the user by causing the display unit 34 to display a message or dialogue indicating that the wireless connection has shifted to an off-state (Step S16a). Then, the control unit 30 carries out the processing of Step S17. By the processing of Step S16a, the user can recognize that the liquid medicine injection device 2 is injecting the liquid medicine and that the user cannot immediately operate the liquid medicine injection device 2 via the controller 3.

After the processing of Step S19, the control unit 30 notifies the user by causing the display unit 34 to display a message or dialogue indicating that the wireless connection has returned to an on-state (Step S19a). Then, the control unit 30 carries out the processing of Step S20. By the processing of Step S19a, the user can recognize that the liquid medicine injection device 2 is not injecting the liquid medicine and that the user can immediately operate the liquid medicine injection device 2 via the controller 3.

Of course, for example, if a setting of an injection program is accepted via the switch unit 33 when the wireless connection is off, the control unit 30 may notify the user by causing the display unit 34 to display a message or dialogue indicating that the wireless connection is off. Thus, the user can recognize that the liquid medicine injection device 2 is injecting the liquid medicine and that the user cannot immediately send the setting of the injection program to the liquid medicine injection device 2 (not until the liquid medicine injection is finished).

Also, in addition to or in place of the notification by the display unit 34, for example, the control unit 30 may gives the buzzer control unit 36 an instruction of an audio signal indicating the on-state or off-state of the wireless connection, so as to cause the buzzer 35 to output a sound indicating the on-state or off-state of the wireless connection. Also, the control unit 30 may notify the user of the on-state or off-state of the wireless connection by causing the light source unit such as an LED to emit light.

If an abnormality in the liquid medicine injection device 2 is detected by the sensor 20 and the sensor control unit 21 during the wireless connection is off, for example, the control unit 10 may give an instruction to the buzzer control unit 23 so as to cause the buzzer 22 to output a sound indicating the occurrence of the abnormality. Thus, the user can be notified of the abnormality even if the wireless connection is off. The control unit 10 may notify the user of the abnormality by causing the light source unit such as an LED to emit light.

Also, if an operation on the stop key of the switch unit 13 is accepted while the wireless connection is off, for example, the control unit 10 may stop the injection program that is being executed and thus stop the liquid medicine injection. Thus, even in the case where the liquid medicine injection cannot be stopped from the controller 3 side because the wireless connection is off, the liquid medicine injection can be stopped by the operation of the liquid medicine injection device 2.

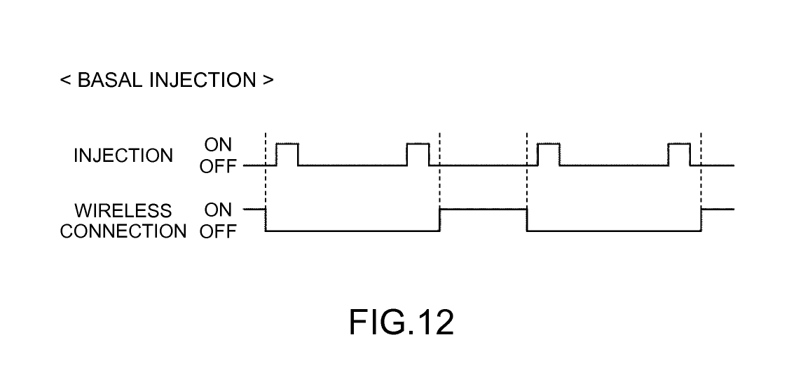

The length of the off-time of the wireless connection in a basal injection is not limited to the example shown in FIG. 8. For example, as shown in FIG. 12 (illustrating a modification of the wireless connection state in a basal injection), one off-time period of the wireless connection may include two injection time periods. In this case, in Step S43 of FIG. 6, two injections are equivalent to the predetermined amount. Similarly, one off-time period of the wireless connection may include three or more injection time periods.

As the length of the off-time of the wireless connection in a basal injection, for example, a plurality of patterns (also referred to as “operation modes”) may be provided as shown in FIGS. 8 and 12 so that one operation mode can be selected. Specifically, the control unit 30 accepts a setting of an operation mode of the off-time of the wireless connection in the basal injection, in addition to the setting of the injection program. Also, the control unit 30 transmits the accepted operation mode to the liquid medicine injection device 2 via the communication unit 31.

The communication unit 11 of the liquid medicine injection device 2 receives the operation mode and stores the operation mode in the storage unit 12. The control unit 10 controls the wireless switching off according to the received operation mode, when executing the basal injection. That is, in Step S40 of FIG. 6, the control unit 10 decides the off-time of the wireless connection corresponding to the received operation mode and transmits the off-time to the controller 3. For example, in the operation mode of FIG. 8, a time period including one injection time period is decided as the off-time. In the operation mode of FIG. 12, a time period including two injection time periods is decided as the off-time.

The control unit 10 also carries out the determination of Step S43 of FIG. 6 according to the received operation mode. For example, if the operation mode of FIG. 8 is selected, the control unit 10 determines the finish of one injection. If the operation mode of FIG. 12 is selected, the control unit 10 determines the finish of two injections.

Thus, the length of the off-time can be set according to the user's demand and the time period when the wireless connection is on can be increased or decreased accordingly. Also, if the time period when the wireless connection is off increases, the power consumption by the liquid medicine injection device 2 can be reduced. Instead of accepting the selection of an operation mode, a setting of a predetermined amount (for example, the number of times) may be accepted and the control unit 10 may decide the off-time or carry out the determination of Step S43 of FIG. 6, according to the set predetermined amount that is set.

The control unit 10 may also switch operation mode, for example, according to the remaining capacity of the power source unit (battery) of the liquid medicine injection device 2. Specifically, the control unit 10 detects the remaining capacity of the power source unit immediately before Step S40 of FIG. 6. The control unit 10 also decides the operation mode corresponding to the remaining capacity of the power source unit. For example, the operation mode may be decided on the basis of the detected remaining capacity, referring to a table that associates remaining battery capacities with operation modes. In the table, for example, an operation mode having more times periods when the wireless connection is off may be associated with a smaller battery remaining capacity. In Steps S40 and S43, the control unit 10 may carry out the processing according to the operation mode that is decided.

Thus, since the liquid medicine injection device 2 increases the time period when the wireless connection is off as the remaining battery capacity decreases, the operation time of the liquid medicine injection device 2 can be extended.

Also, the control unit 10 of the liquid medicine injection device 2 may transmit, for example, only the information that gives an instruction to switch off the wireless connection, to the controller 3, as the wireless off instruction. In this case, as the control unit 30 of the controller 3 receives the wireless off instruction, the control unit 30 decides an off-time of the wireless connection and monitors the lapse of the off-time that is decided, using the timer. The control unit 30 may decide the off-time of the wireless connection on the basis of the content of the injection program that is being executed by the liquid medicine injection device 2, as in the processing of Steps S34 and S40 of FIG. 6.

Moreover, the control unit 10 of the liquid medicine injection device 2 may, for example, refrain from transmitting a wireless off instruction to the controller 3. In this case, the control unit 30 of the controller 3 checks the communication state of the liquid medicine injection device 2 by polling or the like. If communication with the liquid medicine injection device 2 is not available (if the liquid medicine injection device 2 is injecting the liquid medicine, or the like), the control unit 30 decides an off-time of the wireless connection, switches off the wireless connection, and monitors the lapse of the off-time that is decided, using the timer. The control unit 30 may decide the off-time of the wireless connection on the basis of the content of the injection program that is being executed by the liquid medicine injection device 2, as in the processing of Steps S34 and S40 of FIG. 6.

The configurations of the liquid medicine injection device 2 and the controller 3 in FIG. 2 show main components in order to facilitate understanding of the configurations of the liquid medicine injection device 2 and the controller 3. The invention is not limited by the way the components are classified or by the names thereof. The configurations of the liquid medicine injection device 2 and the controller 3 may be divided into a larger number of components according to processing content. Also, one component may be configured to execute a greater amount of processing. The processing by each component may be executed with a single piece of hardware or may be executed with a plurality of pieces of hardware.

The units of processing used in the flowcharts and sequence diagrams explained in FIGS. 5, 6, 9, 10 and 11 are divided according to the main processing content, in order to facilitate understanding of the processing by the liquid medicine injection device 2 and the controller 3. The invention is not limited by the way the units of processing are divided or by the names thereof. The processing by the liquid medicine injection device 2 and the controller 3 can be divided into a greater number of units of processing according to processing content. Also, one unit of processing may be divided to include a greater amount of processing. Moreover, the orders of processing in the flowcharts and sequence diagrams are not limited to the illustrated examples.

The invention is not limited to a liquid medicine injection device using a pump as shown in FIGS. 3A and 3B and FIG. 4 and can also be applied to a liquid medicine injection device using other liquid medicine injection methods such as a cylinder. Also, the invention can be applied not only to wireless connection but also to wired connection.

The entire disclosure of Japanese Patent Application No. 2014-96553, filed May 8, 2014 is expressly incorporated by reference herein.

What is claimed is:

1. A liquid medicine injection device controlled by a controller, the device comprising:
a liquid medicine injection unit;
a first communication unit which communicates with the controller; and
a control unit which switches off the communication by the first communication unit while the liquid medicine injection unit is injecting a liquid medicine.

2. The liquid medicine injection device according to claim 1, wherein
the first communication unit transmits information that gives an instruction to switch off the communication, to the controller.

3. The liquid medicine injection device according to claim 1, wherein
the first communication unit transmits an off-time of the communication to the controller in the case where a liquid medicine injection is started.

4. The liquid medicine injection device according to claim 3, wherein
the control unit switches off the communication in the case of starting a liquid medicine injection and switches on the communication after the liquid medicine injection is finished.

5. The liquid medicine injection device according to claim 1, further comprising
a notification unit which, when an abnormality in the liquid medicine injection device occurs while the communication is off, gives a notification of the occurrence of the abnormality.

6. The liquid medicine injection device according to claim 1, comprising
an operation unit which accepts an operation to stop the injection of the liquid medicine.

7. The liquid medicine injection device according to claim 3, wherein
the off-time is a time period including a time period of one or more continuous liquid medicine injections,
the first communication unit receives a setting of a length of the off-time from the controller, and
the control unit decides the length of the off-time on the basis of the setting.

8. The liquid medicine injection device according to claim 3, wherein
the device operates using a battery as a power source,
the off-time is a time period including a time period of one or more continuous liquid medicine injections, and
the control unit decides a length of the off-time according to a remaining capacity of the battery.

9. A controller for controlling a liquid medicine injection device, the controller comprising:
a second communication unit which communicates with the liquid medicine injection device; and
a control unit which switches off the communication by the second communication unit while the liquid medicine injection device is injecting a liquid medicine.

10. The controller according to claim 9, wherein
the second communication unit receives information that gives an instruction to switch off the communication, from the liquid medicine injection device, and
the control unit switches off the communication if the information that gives the instruction to switch off the communication is received.

11. The controller according to claim 9, wherein
the second communication unit receives an off-time of the communication from the liquid medicine injection device, and
the control unit switches off the communication during the off-time and switches on the communication after the off-time.

12. The controller according to claim 11, comprising
an operation unit which accepts a setting of a length of the off-time,
wherein the second communication unit transmits the setting to the liquid medicine injection device.

13. The controller according to claim 10, comprising
a notification unit that notifies that the communication is shifted to an off-state or returned to an on-state.

14. The controller according to claim 10, comprising:
an operation unit which accepts a setting of the liquid medicine injection device; and
a notification unit which notifies that the communication is in an off-state if the setting of the liquid medicine injection device is accepted while the communication is off.

15. A liquid medicine injection system comprising:
the liquid medicine injection device according to claim 1; and
a controller which controls the liquid medicine injection device;
wherein the controller includes a second communication unit which communicates with the liquid medicine injection device.

16. A liquid medicine injection system comprising:
the controller according to claim 9; and
a liquid medicine injection device controlled by the controller;
wherein the liquid medicine injection device includes
a liquid medicine injection unit, and
a first communication unit which communicates with the controller.

17. A control method for a liquid medicine injection device which communicates with a controller, the method comprising:
switching off the communication with the controller while the liquid medicine injection device is injecting a liquid medicine.
